SEQUENCE LISTING (1 sequence)

SEQ ID NO: 1 - DNA, 6729 bp, Escherichia coli
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagactagtgctgatactcgcattggtgtaacaatctataagtacgacgataactttatgtctgtagtgcgcaaggctattgagcaagatgcgaaagccgcgccagatgttcagctgctgatgaatgattctcagaatgaccagtccaagcagaacgatcagatcgacgtattgctggccaagggggtgaaggcactggccatcaacctggttgacccggcagctgcgggtacggtgattgagaaagcgcgtgggcaaaacgtgccggtggttttcttcaacaaagaaccgtctcgtaaggcgctggatagctacgacaaagcctactacgttggcactgactcaaaagagtccggcattattcaaggcgatttgattgctaaacactgggcggcgaatcagggttgggatctgaacaaagacggtcagattcagttcgtactgctgaaaggtgaaccgggccatccggatgcagaagcacgtaccacttacgtgattaaagaattgaacgataaaggcatcaaaactgaacagttacagttagataccgcaatgtgggacaccgctcaggcgaaagataagatggacgcctggctgtctggcccgaacgccaacaaaatcgaagtggttatcgccaacaacgatgcgatggcaatgggcgcggttgaagcgctgaaagcacacaacaagtccagcattccggtgtttggcgtcgatgcgctgccagaagcgctggcgctggtgaaatccggtgcactggcgggcaccgtactgaacgatgctaacaaccaggcgaaagcgacctttgatctggcgaaaaacctggccgatggtaaaggtgcggctgatggcaccaactggaaaatcgacaacaaagtggtccgcgtaccttatgttggcgtagataaagacaacctggctgaattcagcaagaaaggtaccagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataaaagcttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

(19) United States
(12) Patent Application Publication
(10) Pub. No.: US 2005/0118726 A1
(43) Pub. Date: Jun. 2, 2005

(54) System and method for detecting bioanalytes and method for producing a bioanalyte sensor

(76) Inventors: Jerome S. Schultz, Pittsburgh, PA (US); Kaiming Yi, Pittsburgh, PA (US)

Correspondence Address: Parrish Law Offices, Suite 200, 615 Washington Road, Pittsburgh, PA, 15228, US

(21) Appl. No.: 10/649,433

(22) Filed: Aug. 26, 2003

Related U.S. Application Data
(60) Provisional application No. 60/405,920, filed on Aug. 26, 2002.

(51) Int. Cl. G01N 33/543

(52) U.S. Cl. 436/518

(57) ABSTRACT

The present invention discloses an indicator protein, and a method for making such a fusion protien, having a first binding moiety having a binding domain specific for a class of analytes that undergoes a reproducible allosteric change in conformation when said analytes are reversibly bound; a second moiety and third moiety that are covalently linked to either side of the first binding moiety such that the second and third moieties undergo a change in relative position when an analyte of interest molecule binds to the binding moiety; and the second and third moieties undergo a change in optical properties when their relative positions change and that change can be monitored remotely by optical means. The present invention also discloses a system and method for detecting glucose that uses such a fusion protein in a variety of formats including a subcutaneously and in a bioreactor.

### RELATED APPLICATION

This patent claims priority from provisional application 60/405,920 entitled, “System and Method for Detecting Bioanalytes and Method for Producing a Bioanalyte Sensor,” filed Aug. 26, 2002.

### SEQUENCE LISTING

Applicants submit herewith a Sequence Listing in computer and paper form, in accordance with 37 C.F.R. §1.821-1.825. The content of the paper and computer readable copies of the Sequence Listing submitted in accordance with 37 C.F.R. §1.821(c) and (e) are the same.

### BACKGROUND OF THEE INVENTION

Developing a minimally invasive glucose monitor biosensor to assist in the treatment of diabetes has been a challenge to the analytical community. Despite intensive efforts, mostly based on near infrared spectroscopy (Heise, et. al. 1994), no method is presently available for non-invasively sensing of blood glucose (Tolosa, et al. 1999). Most approaches to this problem have explored minimally invasive techniques. A wide variety of approaches have been developed, including needle-type sensors employing a trilayer coating (Moussy, et al. 1993), microdialysis probes (Keck and Kerner, 1993), amperometric sensors (Pickup, et al., 1993), optical sensors (Rabinnovitch, et al., 1982), calorimetric sensors (Schier, et al., 1988), and fluorescent probes (Schultz, et al., 1982). March (WO 01/13783 A1) shows that the fluorescent probes described by Schultz, et al., 1982, can be incorporated into contact lenses for the measurement of glucose in tear fluid.

De Lorimier, et. al, (2002) review the use of periplasmic proteins that have allosteric properties for biosensor applications, but in the examples given the fluorescent signal was enhanced by the chemical modification of the protein with fluorescent organic chemical species.

Tsien and Miyawaki (U.S. Pat. No. 5,998,204) show that a hybrid fusion protein can be constructed consisting of a donor fluorescent protein moiety, and acceptor fluorescent protein moiety, and a specific analyte binding region, that provides a fluorescent signal that changes with analyte binding. Fehr, et al (2002) describe a maltose indicator protein that changes fluorescence on maltose binding, and later (Fehr, et al 2003) that through directed mutagenesis this protein can be made responsive to glucose in the concentration range of 0.5 to 10 micromolar. Further, although others have attempted to engineer proteins for analyte sensing, see e.g. Lakowicz (U.S. Pat. No. 6,197,534), those individuals have not described a method for making a fusion protein that can be used for such sensing as described herein.

### SUMMARY OF THE INVENTION

The present invention is a method to develop biosensors for bioanalytes by using protein-engineering techniques to integrate signal transduction functions directly into a protein that has specificity for binding the molecule of interest, e.g., glucose binding (Adams, et al. 1991; Brennan, et al. 1995). In the present invention, a receptor protein is selected that undergoes a conformational (allosteric) change accompanying highly specifically binding events to allow one to detect the amount of a selected molecular species in complex mixture (Miyawaki, et al. 1997, Fehr. Et al 2002).

This invention makes such a protein by incorporating optical reporter groups into a fusion protein that contains a specific and reversible binding site (B) for an analyte of interest, such as glucose, in such a manner that the spatial separation between the optical reporter moieties in the protein changes when the ligand binds to section B of the fusion protein. At least one of the optical reporter moieties (A) is a fluorescent protein (such as a green fluorescent protein). The other moiety (C) is a protein that has an absorption spectrum that overlaps the emission of A. The fusion molecule is designed such that the distance between A and C is less than 100 Angstroms so that the hybrid protein exhibits a change in fluorescence energy transfer (FRET) when the analyte binds to B. Moiety C can be a colored protein (such as hemoglobin or chlorophyll), in which case one can monitor the change in emitted fluorescence intensity or fluorescence lifetime of moiety A to monitor the extent of analyte binding to B that is related to the free concentration of analyte in the surrounding fluid. See FIG. 1. Alternatively, moiety C can be another fluorescent protein, selected such that the adsorption spectrum of C overlaps the emission spectrum of A, and in addition where the emission spectrum of C is sufficiently separated from the excitation spectrum of A so that the excitation light does not significantly interfere with the measurement of the emission from C. In this embodiment the measurement of the change in emission intensity from C will reflect the extent of analyte binding to B. See FIG. 1.

One method to make a biosensor based on this new protein is to seal it within a transparent hollow dialysis fiber so as to prevent the leaching out of the indicator protein from the sensor chamber when the sensor is placed in a fluid, but the allowing the analyte to freely exchange between the interior and exterior of the sensor chamber. Also, the porosity of the dialysis fiber is chosen to prevent the intrusion of enzymes into the chamber that could attack the indicator fusion protein. Alternatively, the protein can be immobilized on a solid surface such as fibers, porous particles and gel-like plastics, which can be placed in the fluid(s) of interest. Again, the portion of the solid surface that supports the fusion protein must be freely accessible to analyte residing in the sample fluid.

### BRIEF DESCRIPTION OF THE DRAWINGS

FIG. 1 depicts a schematic representation of allosteric changes when a fusion indicator protein is exposed to glucose.

FIG. 2 depicts the structure of a glucose indicator protein utilizing a selected pair of different green fluorescent proteins wherein GFP represents green fluorescent protein, YFP represents yellow fluorescent protein and GBP represents glucose binding protein.

FIG. 3 depicts the excitation and emission spectra of a fusion glucose indicator proteins containing green fluorescent proteins.

FIG. 4 depicts a glucose indicator protein FRET dependence on glucose concentration.

FIG. 5 depicts a hollow fiber glucose sensor using a glucose indicator protein.

FIG. 6 demonstrates the reversibility of a hollow fiber glucose sensor.

FIG. 7 depicts a preferred embodiment of the instrumentation components for a glucose monitoring system.

FIG. 8 depicts the plasmid DNA sequence of a preferred embodiment of the present invention.

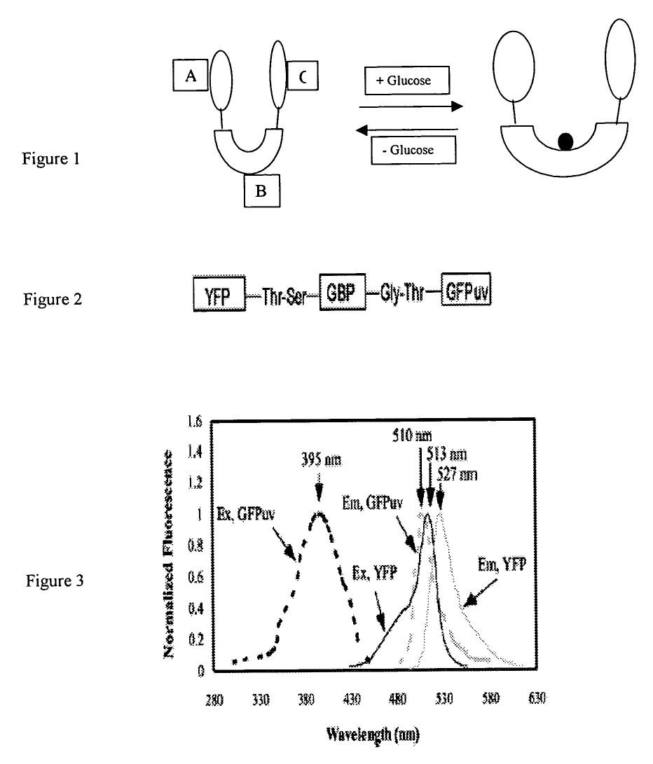

### DETAILED DESCRIPTION OF THE INVENTION

Method of Creating Indicator Fusion Protein

In one preferred embodiment of the present invention, to combine the brightness of fluorescent protein with the targeted molecular indicator, we use a green fluorescent protein isolated from the bioluminescent jelly Aeqorea Victoria (Shimomura, et al., 1962). The cloning of the wild type GFP gene and its subsequent expression in heterologous systems established GFP as a novel genetic reporter system (Prasher, et al. 1992; Chalfire, et al., 1994). Several GFP chromophore variants with shifted excitation and emission wavelengths have been developed by mutagenesis (Heim, et al., 1994; Cormack, et al., 1996), which can serve as donors and acceptors for fluorescence resonance energy transfer (FRET).

As an example of the general class of bioanalyte reporter proteins the present invention presents a new hybrid glucose binding protein that provides changes in fluorescence when glucose binds. This construct utilizes the conformational change-induced FRET between a donor GFP (moiety A) and an acceptor YFP (moiety C) fused to the amino and carboxy termini of a Glucose Binding Protein (moiety B) isolated from E. coli K12 (Scholle, et al. 1987). This fusion molecule has four domains. Two domains involving the Glucose Binding Protein (GBP) that are used to bind the glucose and cause the change in the conformation of the GBP which is interposed between the two fluorescent proteins. In addition when the fluorophore domain in the GFP is excited by light, the emitted fluorescent energy can be transferred to the fluorophore domain in the YFP when the two fluorophores are within 50 angstroms of each other. After the glucose binds to the protein, the rearrangement of the flap region located in one side of the hinge β-sheet of the GBP occurs, which gives rise to the conformation change. The change in the conformation of the GBP upon the binding of the glucose, in turn, alters the relative position of the GFP donor and YFP acceptor which gives rise to change in FRET and a change in the fluorescence lifetime of GFP. The structure of such a glucose indicator protein is shown in FIG. 2, and its preparation is described in Ye and Schultz (2003).

The affinity constant of the binding protein for the analyte must be in a range so that one achieves a variation in the saturation of the binding site over the range of concentrations of the analyte in the sample of interest. To meet this requirement the structure of binding moiety (B) can be modified by genetic engineering techniques (e.g. site directed mutagenesis, error prone PCR) to seek a protein with the desired binding affinity for the analyte.

To achieve a measurable signal when glucose binds to GBP in the present invention two fluorescent proteins are fused, one to each end of the GBP. This construct utilizes a Green Fluorescent Protein mutant (YFP) (with a maximum excitation at 513 nm and maximum emission at 527 nm) and a green GFPuv (with a maximum excitation at 395 nm and maximum emission at 510 nm). The fusion protein was designated as YFP-GBP-GFP. The amino acids sequences of the boundary region between fusion proteins were optimized to achieve a correct and stable folding of the fusion protein. (FIG. 2).

The fusion protein YFP-GBP-GFP has two emission peaks at 510 nm and 527 nm, respectively when excited at 395 nm (FIG. 3). The appearance of emission spectrum at 510 nm shows the fluorescence resonance energy transfers from the GFP donor (emitted at 510 nm when excited at 395 nm) to the YFP acceptor that has emission spectrum at 527 nm when excited at 510 nm.

A special feature of this sensor structure is that there is direct transduction of a fluorescent signal on introduction of the analyte, whereas in previous sensors developed by Schultz, et al (Schultz, et al. 1982) a competing ligand such as FITC-dextran was required to generate a fluorescent signal.

Glucose Transduction Properties of the Preferred Embodiment Fusion Protein YFP-GBP-GFP.

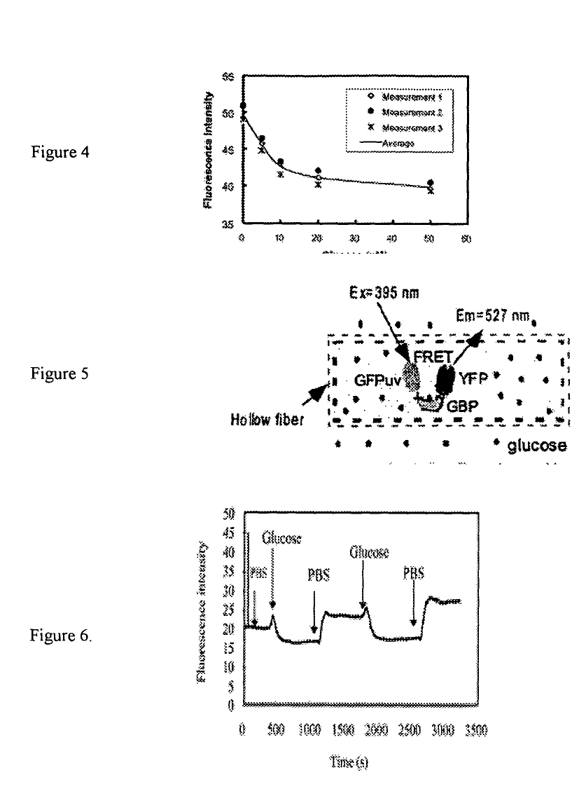

The reduction of fluorescence was observed with the addition of glucose (from 0-0.5 micromolar of final concentration) to the protein solution of YFP-GBP-GFP (FIG. 4). The glucose binding was determined by measuring the changes in FRET on a luminescence spectrometer at room temperature. Glucose was titrated into the protein solution and the fluorescence was determined at Ex=395 nm; Em=527 nm for YFP-GBP-GFP.

Use of the Biosensor to Detect Glucose

The present invention discloses how the induction of conformational change in a protein can be exploited to construct integrated signal transduction function that converts a ligand binding event into a change in a fluorescence signal. This change in emitted fluorescence could be used for the detection of glucose concentration by a device such as a implantable hollow fiber sensor as illustrated in FIG. 5.

A fusion protein is filled into the hollow fiber that is sealed on both ends. In one preferred embodiment approximate dimensions of the hollow fiber sensor are 0.5 mm diameter and 1 cm in length. Glucose from the surrounding media can freely enter the chamber through the dialysis membrane and interact with the fusion protein. Because the binding to the fusion protein is reversible, if the glucose content of the surrounding fluid drops the glucose concentration inside the chamber will also drop causing some dissociation of the glucose from the fusion protein and a change in the protein's conformation. The sensor fiber was placed in solutions containing various concentrations of glucose.

The hollow dialysis fiber had pores with a 1 KDa molecule weight cut off. This retained the YFP-GBP-GFP protein within the fiber and also allows glucose to exchange freely between the fiber lumen and the external solution. The hollow fiber was set up inside a flow cell cuvette (Perkin-Elmer) for measuring the extent of fluorescence quenching upon exposure of the hollow fiber sensor to various concentrations of glucose in the external solution. FIG. 6 shows a typical response of the sensor to the glucose. A sugar-free phosphate buffered saline was used to produce a base line for the sensor.

Clearly, the change of the conformation of the fusion protein YFP-GBP-GFP due to the binding of glucose is reversible as evidenced by the changes in measured fluorescence. Fluorescence is enhanced in the absence of glucose and reduced in the presence of glucose.

Instrumentation

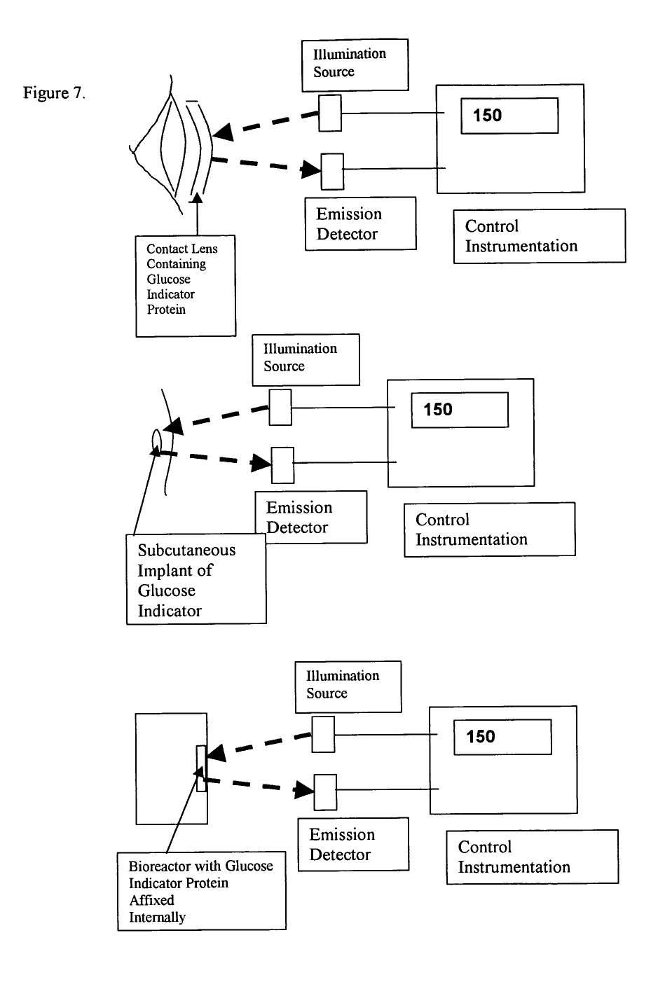

A preferred embodiment of the instrumentation set up to measure glucose concentrations in various media is depicted in FIG. 7.

Alternate methods of detecting the binding of the analyte (e.g., glucose) to the fusion protein are available, such as monitoring changes in the fluorescence lifetime of the fluorescent moieties in the hybrid fusion protein (YFP or GFP) as illustrated by the work of Lakowicz's group by modulating the excitation light source at 100 MHz (Tolosa, et al. 1999).

Nucleic Acid Sequence for Plasmid of Glucose Indicator Protein

The present invention also discloses the plasmid structure encoding YFP-GBP-GFP in FIG. 8. Retroviral vectors can be used for integrating a target gene in the genome of a variety of cells including human and mouse cells (Hawley, et al 1994). Integration of target gene in the genome of cells is important to development of an intracellular glucose biosensor because it allows introducing a “glucose biosensor gene” into cells so that a cell can produce its own intracellular biosensor for continuously glucose monitoring.

1. An indicator protein comprising:
a) a first binding moiety having a binding domain specific for a class of analytes that undergoes a reproducible allosteric change in conformation when said analytes are reversibly bound;
b) a second moiety and third moiety that are covalently linked to either side of said first binding moiety in a manner that said second and third moieties undergo a change in relative position when said analyte molecule binds to said first binding moiety; and
c) said second and third moieties interact to produce a change in optical properties when the relative positions of said second and third moieties change, wherein said change can be monitored remotely by optical means.

2. The protein of claim 1, wherein
a) said first binding moiety is a protein that undergoes allosteric conformational changes when glucose reversibly binds;
b) said second moiety is a fluorescent protein;
c) said third moiety is a protein that has an absorption spectrum that overlaps the emission spectrum of said second moiety;
d) the fluorescent energy transfer changes from said second moiety to said third moiety when glucose binds to said first binding moiety; and
e) hybrid fusion joins said first, second and third moieties.

3. The protein of claim 2 wherein said third moiety is a fluorescent protein that can emit light when fluorescent energy transfers from said second moiety and said third moiety.

4. The protein of claim 2, wherein
a) said first binding moiety is a glucose binding protein from E. coli;
b) said second moiety is EBFP; and
c) said third moiety is hemoglobin.

5. The protein of claim 2, wherein
a) said first binding moiety is a glucose binding protein from E. coli;
b) said second moiety is YFP; and
c) said third moiety C is GFP.

6. The protein of claim 5 having the plasmid sequence shown in FIG. 8.

7. A biosensing system for glucose comprising:
a) a biosensor element consisting of a protein
i. having a first binding moiety, which is a glucose binding protein from E. coli, having a binding domain specific for glucose that undergoes a reproducible allosteric change when glucose is reversibly bound;
ii. having a second moiety and third moiety that are covalently linked to either side of said first binding moiety in a manner such that they change in relative position when glucose binds to said first binding moiety and wherein said second moiety and said third moiety interact to produce a change in optical properties when their relative positions change wherein said optical properties change can be monitored remotely by optical means; and
iii. that is immobilized to a solid surface or retained within a permeable capsule;
b) the placement of said biosensor element in contact with a fluid of interest so that said biosensor element can be illuminated and emitted light detected; and
c) an optical system for illumination of said biosensor element and detection of emitted radiation.

8. A biosensing system for glucose of claim 7 wherein said second moiety is EBFP and said third moiety is hemoglobin.

9. A biosensing system for glucose of claim 7 wherein said second moiety is YFP and said third moiety is GFP.

10. A biosensing system for glucose of claim 8 wherein said contact with a fluid of interest is subcutaneous.

11. A bionsensing system for glucose of claim 9 wherein said contact with said fluid of interest is subcutaneous.

12. A biosensing system for glucose of claim 8 wherein said contact with a fluid of interest occurs through a bioreactor.

13. A biosensing agent for glucose of claim 9 wherein said contact with a fluid of interest occurs through a bioreactor.

14. A biosensing system of claim 7 further comprising an instrument to measure changes in the fluorescence properties of said second moiety and said third moiety.

15. A method for noninvasively measuring glucose within cells wherein
a. plasmid coding for a protein having
i. a first binding moiety having a binding domain specific for a class of analytes that undergoes a reproducible allosteric change in conformation when said analytes are reversibly bound;
ii. a second moiety and third moiety that are covalently linked to either side of said first binding moiety in a manner that said second and third moieties undergo a change in relative position when said analyte molecule binds to said first binding moiety; and
iii. said second and third moieties undergo a change in optical properties when the relative positions of said second and third moieties, wherein said change can be monitored remotely by optical means is introduced into cells;
b. said protein is expressed in the cells; and
c. said changes in fluorescence properties are measured optically by an instrument having an optical system for illumination and detection of emitted radiation.

16. A method for noninvasively measuring glucose within cells of claim 15 wherein said second moiety is YFP and said third moiety is GFP.

17. A method for noninvasively measuring glucose within cells of claim 15 wherein said second moiety is EBFP and said third moiety is hemoglobin.
